(19) United States
(12) Patent Application Publication
(10) Pub. No.: US 2008/0268025 A1
(43) Pub. Date: Oct. 30, 2008

(54) Compositions Useful for Preventing Pain and Soreness Resulting from Exercise and Methods of Use

(76) Inventor: Donald Spector, New York, NY (US)

Correspondence Address: DIEHL SERVILLA LLC, 77 BRANT AVE, SUITE 210, CLARK, NJ, 07066, US

(21) Appl. No.: 11/748,115

(22) Filed: May 14, 2007

Related U.S. Application Data
(60) Provisional application No. 60/913,895, filed on Apr. 25, 2007.

(51) Int. Cl. A61K 9/08; A61K 31/60

(52) U.S. Cl. 424/439; 514/165

(57) ABSTRACT

Provided are beverages containing one or more anti-inflammatory agents, derivatives thereof, prodrugs thereof, salts thereof, or combinations thereof. Additional components include, but are not limited to, electrolytes, minerals, vitamins, flavorings, antacids, buffers, dissolution enhancers and solubility enhancers. Additionally, a beverage mix containing an anti-inflammatory agent is disclosed. Furthermore, a method for preventing or mitigating pain or soreness is disclosed.

### CROSS-REFERENCE TO RELATED APPLICATIONS

This application claims the benefit of priority under 35 U.S.C. § 119(e) to U.S. Provisional Application Ser. No. 60/913,895, filed Apr. 25, 2007, which is incorporated herein by reference.

### TECHNICAL FIELD

The present invention pertains to compositions to prevent or mitigate soreness, inflammation and pain resulting from strenuous physical exertion. Additionally, methods are provided for preventing and mitigating soreness, inflammation and pain resulting from strenuous physical exertion.

### BACKGROUND

During periods of strenuous physical exertion, the body undergoes many physiological reactions. For example, as a person exercises, their body temperature elevates; sweating increases as a means of cooling the body; the heart beats faster to supply more oxygen to the muscles; and digestion slows to preserve all available energy for muscle usage. These physiological responses can have repercussions—cramps, muscle soreness and pain.

Even though it may not be noticed, people sweat constantly. The purpose of sweating is to regulate body temperature by removing excess body heat which is generated by muscles and metabolic processes. The amount of sweat depends on, among other things, the amount of physical exertion being done by the body. During periods of strenuous activity more sweat is generated because more heat is generated. The sweat reaches the surface of the skin where it is evaporated, causing a decrease in temperature at the point where it was on the skin. This is known as evaporative cooling.

The primary components of sweat include water, sodium chloride, potassium, Vitamin C, uric acid, urea, ammonia and lactic acid, amongst other components. These chemicals are brought to the surface of the skin, where the water evaporates, leaving the chemicals behind. The loss of too much salt and water from the body can cause dehydration, kidney failure, heat stroke and other problems. Therefore, it is important to consume fluids to remain hydrated.

However, too much water can result in water intoxication, also called hyperhydration or water poisoning. Hyperhydration occurs when the amount of electrolytes within the body are outside of the safe ranges. This danger is particularly prevalent in marathon runners who consume only water while running. Therefore, most long distance runners consume beverages which contain electrolytes levels which closely match the amount of electrolytes lost through sweating.

Sports drinks have been developed to allow a person to rehydrate while minimizing the risk of hyperhydration. Sports drinks typically contain water, electrolytes, nutrients and sugars to replace those lost during sweating. The level of these components is such that the sports drink is isotonic with sweat.

Physical exertion and exercise can cause muscle soreness and pain. Muscles feel sore because they have microscopic tears which need to heal. This soreness, which usually reaches its maximum level a couple days after exercise, will generally resolve itself in about a week. This is referred to as Delayed Onset Muscle Soreness (DOMS). Although the soreness associated with DOMS will dissipate on its own, it would be preferable to prevent it from occurring at all.

Taking an analgesic, such as aspirin, can help ease pain. For example, people will take aspirin or ibuprofen to stop headaches or to stop pain from muscle soreness that could result from exercise. This is referred to as “chasing the pain,” because the drug is employed to ease pain which already exists. It is, therefore, very desirable to develop a means of preventing inflammation and/or pain, so that it is not necessary to chase it. An anti-inflammatory drug, such as a non-steroidal anti-inflammatory drug (NSAID), can be effective at a lower dose when no pain is present. Once pain has been developed, a larger amount of the drug will be necessary to reduce inflammation.

### SUMMARY OF THE INVENTION

One or more embodiments of the invention pertain to a beverage containing an anti-inflammatory compound, a derivative thereof, a prodrug thereof, a salt thereof, or combinations thereof. Reference to a derivative thereof, a prodrug thereof, a salt thereof, or combinations thereof means pharmaceutically acceptable forms of the same. In various embodiments, the beverage can be water, or a sports drink. In other embodiments, the beverage contains excipients, such as, electrolytes, vitamins, minerals, antacids, buffers and flavorings. In further embodiments, the anti-inflammatory agent is present at lower doses than would otherwise be required for pain relief at, for example, the onset of pain. It should be understood that the present invention is not limited to anti-inflammatory compounds which are also analgesics. In additional embodiments, the beverage contains more than one anti-inflammatory agent. In other embodiments, the beverage contains excipients which can increase the anti-inflammatory's solubility or rate of dissolution. In still further embodiments, the beverage contains excipients which can decrease the taste associated with the anti-inflammatory agent.

Another embodiment of the invention pertains to a beverage mix which contains an anti-inflammatory agent. The agent being present in sufficient amount as to act as an anti-inflammatory when the mix is dissolved or dispersed in a prescribed amount of liquid. In one or more embodiments, the beverage mix contains excipients which act as antacids, buffers, flavorings, dissolution or solubility enhancers, suspending agents, or mask the taste of the agent. In further embodiments, the beverage mix contains more than one anti-inflammatory agent.

A further embodiment of the invention is a method for preventing pain and soreness by drinking a beverage which has anti-inflammatory agent. The beverage may be consumed before and/or during physical activity.

### DETAILED DESCRIPTION OF THE INVENTION

Before describing several exemplary embodiments of the invention, it is to be understood that the invention is not limited to the details of construction or process steps set forth in the following description. The invention is capable of other embodiments and of being practiced or being carried out in various ways.

As used in this specification and the appended claims, the singular forms “a”, “an” and “the” include plural referents unless the context clearly indicates otherwise. Thus, for example, reference to “an anti-inflammatory agent” includes a mixture of two or more anti-inflammatory agents, and the like.

Where the term “anti-inflammatory agent” or “anti-inflammatory” is used, it should be understood to include derivatives, prodrugs, salts, or combinations of the same of the anti-inflammatory as well.

The use of the term “excipient” refers to any chemical other than the anti-inflammatory or water. Typically, an excipient is an inactive ingredient which is used as a carrier for an active ingredient, anti-inflammatory in this case. However, some of the additional compounds used herein are efficacious for some purpose other than as an anti-inflammatory (i.e., antacids). These components, for example, while not used as a carrier, are included in the definition of “excipient” as it is used.

A non-exclusive list of anti-inflammatory agents which can be employed with the various embodiments of the invention include over-the-counter drugs: ibuprofen, naproxen, naproxen sodium, acetylsalicylic acid (aspirin), ketoprofen, choline salicylate, magnesium salicylate. Embodiments of the present invention can also use prescription drugs: sulindac, oxaprozin, salsalate, prioxicam, indomethacin, etodolac, meloxicam, nabumetone, ketorolac, and COX-2 inhibitors. Other anti-inflammatory agents can include herbal products. Still other anti-inflammatory agents, can be used in the various embodiments, and would be known to one skilled in the art.

The term “sports drink,” as used herein, includes Sports Drinks, Energy Drinks and any beverage which contains electrolytes, vitamins, minerals, caffeine, sugars, proteins or any combination of these ingredients. Typically, Sports Drinks replenish water, electrolytes, sugars and nutrients. Sports Drinks are often isotonic, but can be hypertonic or hypotonic depending upon the needs of the athlete. Examples of Sports Drinks include, but are not limited to, Gatorade®, Powerade®, Accelerade® and Lucozade Sport®. Energy Drinks, or Power Drinks, typically contain sugars and caffeine, providing a short boost of energy. Commercial products Red Bull®, Lucozade®, Mountain Dews and AMPS would fall into the Energy Drink category. It should be understood that the use of the term “sports drink” should not be taken as being one single formulation of these commercially available products, although they can be used, but should encompass the general category to which these products belong.

Use of the terms “pharmaceutical”, “pharmaceutically” and “pharmaceutically acceptable” refer to ingredients which are commonly used in products that have to or had to undergo U.S. Food & Drug Administration (FDA) approval, or that fall under the FDA purview or review. Herbal ingredients, natural and synthetic, are excluded from the definition of pharmaceutical as used herein.

One or more embodiments of the invention is a beverage comprising an anti-inflammatory agent. In various embodiments, more than one anti-inflammatory agent can be used at the same time. The beverage of one embodiment comprises water with an anti-inflammatory agent added. In another embodiment, the beverage comprises a sports drink with an anti-inflammatory agent added.

In multiple embodiments, the beverage contains various excipients, in addition to the anti-inflammatory agent. These can be electrolytes, vitamins, minerals, buffers, antacids, proteins, surfactants, flavorings and other compounds.

Electrolytes, as used herein, include, but are not limited to, sodium (Na⁺), potassium (K⁺), lithium (Li⁺), calcium (Ca²⁺), magnesium (Mg²⁺), chloride (Cl⁻), phosphate (PO₄³⁻), carbonate (CO₃²⁻), nitrate (NO₃²⁻), nitrite (NO₂⁻), and sulfate (SO₄²⁻) ions. Additional compounds which can be employed as electrolytes would be known to one skilled in the art.

Some embodiments contain one or more vitamins, in addition to the anti-inflammatory agent. Typical vitamins which can be used are the water soluble vitamins: Vitamin B₁ (thiamine), Vitamin B₂ (riboflavin), Vitamin B₃ (niacin), Vitamin B₅ (pantothenic acid), Vitamin B₆ (pyridoxine), Vitamin B₇ (biotin), Vitamin B₉ (folic acid), Vitamin B₁₂ (cyanocobalamin), Vitamin C (ascorbic acid), adenine and adenylic acid; the fat soluble vitamins Vitamin A (retinoids), Vitamin D (ergocalciferol, cholecalciferol, doxercalciferol), Vitamin E (tocopherol) and Vitamin K (naphthoquinone). It should be emphasized that this is not an exclusive list of vitamins which can be used with the various embodiments of this invention. Other vitamins will be known to those of skill in the art.

In still further embodiments, the anti-inflammatory beverage contains one or more minerals. A non-exhaustive list of minerals for use in the various embodiments include: bismuth, boron, calcium, chromium, cobalt, copper, fluorine, germanium, gold, indium, iodine, iridium, iron, magnesium, manganese, molybdenum, nickel, phosphorus, platinum, potassium, rhodium, rubidium, selenium, silicon, silver, sodium, sulfur, tellurium, titanium, tungsten, vanadium, and zinc. Most minerals will also act as electrolytes and can serve both roles in the various embodiments. Other minerals which can be used will be known to those of skill in the art.

Further embodiments include excipients which act as antacids and buffers. Other embodiments include flavorings. Typical antacids, buffers and flavorings would be known to those skilled in the art.

Still other embodiments include herbal excipients, such as, glucosamine, chondroitin, ginseng, glutamine, japanese honeysuckle, royal jelly, saw palmetto, wolfberry, yohimbine. Other herbal components can be included in the various embodiments, and would be known to one skilled in the art.

In further embodiments, the anti-inflammatory agent is also an analgesic, and is present at lower doses than would be required for pain relief. Not all anti-inflammatory compounds are effective in pain relief, just as not all analgesics have anti-inflammatory effects. One or more embodiments contain analgesic anti-inflammatories present at up to 25%, 50% and 75% of the pain relieving dosage at, for example, the onset of pain. One skilled in the art understands that dosage generally relates to the amount of active ingredient needed to be effective to reduce pain in a person of a certain weight. It should be understood that the present invention is not limited to anti-inflammatory compounds which are also analgesics.

The anti-inflammatory agent is preferably provided in an amount at which the agent is effective. Some examples of preferred embodiments including effective amounts are provided.

In an embodiment, the active ingredient provides acetylsalicylic acid in the range of about 10 mg to about 975 mg. In this way, the active ingredient can comprise any form of acetylsalicylic acid, a derivative, a prodrug, or a salt thereof in an amount needed to provide the active ingredient in the desired range. When the active pharmaceutical agent is acetylsalicylic acid, the agent is preferably present in the range of about 10 mg to about 975 mg or when a pharmaceutically acceptable salt thereof is used, it is present in an equivalent amount of the salt. Alternatively, the acetylsalicylic acid can be present in the amount of up to about 243 mg. In accordance with another embodiment of the present invention, the acetylsalicylic acid can be present in the amount of up to about 487 mg. In accordance with yet another embodiment of the present invention, the acetylsalicylic acid can be present in the amount of up to about 730 mg.

In an embodiment, the active ingredient provides naproxen in the range of about 5 mg to about 500 mg. In this way, the active ingredient can comprise any form of naproxen, a derivative, a prodrug, or a salt thereof, in an amount needed to provide the active ingredient in the desired range. When the active pharmaceutical agent is naproxen, the agent is preferably present in the range of about 5 mg to about 500 mg or when a pharmaceutically acceptable salt thereof is used, it is present in an equivalent amount of the salt. Alternatively, the naproxen can be present in the amount of up to about 125 mg. In accordance with another embodiment of the present invention, the naproxen can be present in the amount of up to about 250 mg. In accordance with yet another embodiment of the present invention, the naproxen can be present in the amount of up to about 375 mg.

In an embodiment, the active ingredient provides ibuprofen in the range of about 4 mg to about 400 mg. In this way, the active ingredient can comprise any form of ibuprofen, a derivative, a prodrug, or a salt thereof in an amount needed to provide the active ingredient in the desired range. When the active pharmaceutical agent is ibuprofen, the agent is preferably present in the range of about 4 mg to about 400 mg or when a pharmaceutically acceptable salt thereof is used, it is present in an equivalent amount of the salt. Alternatively, the ibuprofen can be present in the amount of up to about 100 mg. In accordance with another embodiment of the present invention, the ibuprofen can be present in the amount of up to about 200 mg. In accordance with yet another embodiment of the present invention, the ibuprofen can be present in the amount of up to about 300 mg.

In an embodiment, the active ingredient provides ketoprofen in the range of about 0.1 mg to about 75 mg. In this way, the active ingredient can comprise any form of ketoprofen, a derivative, a prodrug, or a salt thereof in an amount needed to provide the active ingredient in the desired range. When the active pharmaceutical agent is ketoprofen, the agent is preferably present in the range of about 0.1 mg to about 75 mg or when a pharmaceutically acceptable salt thereof is used, it is present in an equivalent amount of the salt. Alternatively, the ketoprofen can be present in the amount of up to about 18 mg. In accordance with another embodiment of the present invention, the ketoprofen can be present in the amount of up to about 37 mg. In accordance with yet another embodiment of the present invention, the ketoprofen can be present in the amount of up to about 56 mg.

Some anti-inflammatory compounds are not very soluble in water, or take a long time to dissolve. In other embodiments, the beverage contains excipients which can increase the solubility or dissolution rate of the anti-inflammatory agent. These compounds include, but are not limited to, compounds that encapsulate the anti-inflammatory agent, surfactants, liposomes and compounds which will form micelles. Appropriate compounds for this purpose would be known to those skilled in the art. One or more embodiments may include suspending agents used to aid in dispersing the anti-inflammatory agent. In one or more embodiments, a suspending agent includes a surfactant. Other suspending agents include, but are not limited to, xanthan gum and polyoxyethylene sorbitan monooleate (polysorbate 80).

In still further embodiments, the beverage contains excipients which can counteract the taste associated with the anti-inflammatory. Many anti-inflammatory compounds have a bitter taste associated with them. Addition of a compound to mask this taste can be employed. A common interaction to mask the taste is to encapsulate the anti-inflammatory molecule within in a micelle. Although it should be understood that this is not the only means for decreasing the taste associated with the anti-inflammatory. Appropriate compounds, and techniques, for this purpose would be known to those skilled in the art.

Other embodiments of the invention pertain to a beverage mix which contains an anti-inflammatory agent as the active component. The anti-inflammatory agent is preferably present in an amount which is sufficient to be effective when the mix is dissolved in a prescribed amount of liquid. In some embodiments the mix is in a dry powder form which can be dissolved in a specified amount of a liquid. In other embodiments, the mix is a concentrated liquid which can be diluted in a specified amount of another liquid. These embodiments can be packaged in individual serving packages which can be dissolved, diluted or dispersed in either a standard 0.5 L bottle of water or in a sports drink.

In one or more embodiments, the beverage mix includes excipients which will act as antacids, buffers, flavorings, increase dissolution rate, increase solubility, or mask the taste of the agent. In further embodiments, the beverage mix contains more than one anti-inflammatory agent.

Still further embodiments of the invention pertain to a method for preventing pain and soreness by drinking a beverage which has pre-mixed anti-inflammatory components. The amount of anti-inflammatory agent used in can be lower than the normal dosage, or equal to the normal dosage. Reference to normal dosage means the dosage of the anti-inflammatory agent that is usually available to a consumer, for example, an over-the-counter dosage. The beverage may be consumed prior to, or during physical activity, or both. In other embodiments, the method includes the steps of dissolving or dispersing the beverage mix in a suitable amount of liquid (i.e., a 0.5 L bottle of water, or a bottle of sports drink). In further embodiments, the method includes the steps of diluting a beverage mix concentrate in a suitable amount of liquid.

Accordingly, while the present invention has been disclosed in connection with various embodiments thereof, it should be understood that other embodiments might fall within the spirit and scope of the invention, as defined by the following claims.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. An anti-inflammatory beverage to be consumed by a person, comprising:
a sports drink; and
an active ingredient;
wherein the active ingredient comprises an anti-inflammatory pharmaceutical agent, derivatives thereof, prodrugs thereof, salts thereof, or combinations thereof.

2. The beverage of claim 1, wherein the beverage comprises one or more electrolytes.

3. The beverage of claim 1, wherein the beverage comprises one or more vitamins.

4. The beverage of claim 1, wherein the beverage comprises one or more minerals.

5. The beverage of claim 1, wherein the beverage comprises one or more flavorings.

6. The beverage of claim 1, wherein the beverage comprises a protein.

7. The beverage of claim 1 wherein the active ingredient is selected from the group consisting of acetylsalicylic acid, naproxen, ibuprofen, ketoprofen, choline salicylate, magnesium salicylate, derivatives thereof, prodrugs thereof, salts thereof, or combinations thereof.

8. The beverage of claim 1 wherein the active ingredient provides acetylsalicylic acid in the range of about 10 mg to about 975 mg.

9. The beverage of claim 1 wherein the active ingredient provides naproxen in the range of about 5 mg to about 500 mg.

10. The beverage of claim 1 wherein the active ingredient provides ibuprofen in the range of about 4 mg to about 400 mg.

11. The beverage of claim 1 wherein the active ingredient provides ketoprofen in the range of about 0.1 mg to about 75 mg.

12. The beverage of claim 1 wherein the active ingredient comprises two or more of acetylsalicylic acid, naproxen, ibuprofen, ketoprofen, choline salicylate, magnesium salicylate, derivatives thereof, prodrugs thereof, salts thereof, or combinations thereof.

13. The beverage of claim 1 further comprising one or more of an antacid, a buffering agent, or a flavoring agent.

14. The beverage of claim 1 further comprising at least one excipient for increasing the solubility of the active ingredient.

15. The beverage of claim 1 further comprising at least one surfactant.

16. The beverage of claim 1 further comprising at least one excipient for masking the taste associated with the active ingredient.

17. The beverage of claim 1, wherein the active ingredient is also a pain reliever, and is present at 75% of the normally prescribed dosage when used as a pain reliever.

18. The beverage of claim 17, wherein the active ingredient is also a pain reliever, and is present at 50% of the normally prescribed dosage when used as a pain reliever.

19. The beverage of claim 18, wherein the active ingredient is also a pain reliever, and is present at 25% of the normally prescribed dosage when used as a pain reliever.

20. A method of preventing pain or soreness comprising drinking a beverage having a liquid and a pre-mixed, low-dosage anti-inflammatory agent prior to engaging in physical activity and/or while engaging in physical activity.

21. The method of claim 20, further comprising dissolving or dispersing a dry powder anti-inflammatory beverage mix in a suitable amount of the liquid to form the beverage.

22. The method of claim 20, further comprising diluting an anti-inflammatory beverage mix concentrate in a suitable amount of the liquid.
